(19) United States
(12) Patent Application Publication
(10) Pub. No.: US 2011/0202074 A1
(43) Pub. Date: Aug. 18, 2011

(54) DEVICES AND METHODS FOR DEPLOYING MEDICAL SUTURES

(76) Inventors: Paul A. Talmo, Jamaica Plain, MA (US); Michael Lovuolo, Marshfield, MA (US); Thomas A. Jellison, Warwick, RI (US)

(21) Appl. No.: 12/937,154

(22) PCT Filed: Apr. 9, 2009
(86) PCT No.: PCT/US09/40024

§ 371 Date: Apr. 28, 2011

Related U.S. Application Data
(60) Provisional application No. 61/043,561, filed on Apr. 9, 2008.

(51) Int. Cl. A61B 17/04

(52) U.S. Cl. 606/145; 606/228

(57) ABSTRACT

The invention relates to a medical suture (100) and devices and methods for deploying a medical suture. One embodiment of the invention relates to a suture deployment device (800) and method of use, wherein the suture deployment devices includes a housing (805), a substantially hollow elongate needle (600), a suture storage cartridge (700), a push rod (740) adapted to retractably extend through the hollow elongate needle, and a deployment mechanism (815) for retractably extending the push rod though the hollow elongate needle, wherein the push rod is adapted to eject a loaded suture from a distal end of the hollow elongate needle when extended.

### CROSS-REFERENCE TO RELATED APPLICATIONS

This application claims priority to and the benefit of U.S. provisional patent application Ser. No. 61/043,561 filed Apr. 9, 2008, the disclosure of which is being incorporated herein by reference in its entirety.

### TECHNICAL FIELD

The invention generally relates to devices and methods for medical suturing. More particularly, the invention relates to devices and methods for quickly and safely deploying medical sutures for use, for example, in hernia or pelvic prolapse repair.

### BACKGROUND

Urinary incontinence and pelvic prolapse are conditions that arise from hernias which have developed in the vagina and which result in the loosening of various structures. When the area of the bladder neck is weakened, the resulting movement may cause a loss of the seal and therefore a failure of the normal continence mechanism, resulting in possible urinary leakage. When the various walls of the vagina weaken, the underlying structures (such as, for example, the bladder, rectum, uterus, and small bowel) bulge into the vagina, causing the vaginal skin to prolapse. Various terms (such as, for example, cystocele, rectocele, uterine prolapse, and enterocele) are used to describe which of the underlying structures are prolapsing.

It is known that female incontinence and pelvic prolapse can be corrected by surgical restoration of the urethrovesical junction to its proper orthotopic position, for example by suspending, stabilizing, and/or compressing the bladder neck to achieve a position wherein incontinence will be avoided but normal urinary function will not be hindered.

In general, the basic principles of prolapse repair surgery is often to re-create support of the various hernias by using a sling material, such as a mesh or hammock-type support element, to create a layer of support. This often involves placing the sling material under the bladder neck and attaching it to some strong structure. Typically, a layer of connective tissue (e.g. rectus fascia) just underneath the fat of the abdominal skin has been used for the attachment structure. An incision is made in the vagina under the bladder neck and then a mesh or suture material is passed upwards through an incision in the abdomen and anchored to the rectus fascia. These procedures, however, have generally been accomplished through an abdominal or vaginal incision which involves extensive dissection and significant time in order to carry out the procedure.

Similar surgical techniques can also be used in the surgical treatment and repair of hernias. A hernia develops when the outer layers of the abdominal wall weaken, bulge, or actually rip. The hole in this outer layer allows the inner lining of the cavity to protrude and to form a sac. Any part of the abdominal wall can develop a hernia. The most common site for hernias is the groin (i.e. an inguinal hernia).

### SUMMARY OF THE INVENTION

There is a need to provide devices and methods that can be used to decrease the required dissection and surgery time while increasing the safety and efficiency of surgical procedures such as, for example, hernia repair or prolapse repair surgery. The suture deployment devices and methods described herein may enable hernia or prolapse repair procedures, and other appropriate medical procedures, to be done faster, quicker, and safer. They may also allow potential migration of these invasive procedures, which are currently being performed in the operating room under anesthesia, to a completely non-invasive office-based procedure, wherein no incision is necessary.

In one embodiment, the present invention may provide a suture and a suture deployment device, and associated methods of use. The invention allows for the pinpoint attachment of a support element through minimal incisions with greater safety, speed, and efficacy. In addition, such devices and methods may be easily and consistently operated by a user and provide a broad-based flexibility for use in various other medical applications.

One aspect of the invention includes a medical suture. The medical suture may include an elongate suture wire, a first anchoring element connected to a first end of the elongate suture wire, and a second anchoring element connected to a second end of the elongate suture wire. In one embodiment, the elongate suture wire may include a material selected from the group consisting of a polymer, a metal, a plastic, a fabric, and combinations thereof. The polymer may be an absorbable polymer, such as, but not limited to, an absorbable polylactic acid (PLA) or an absorbable polyglycolic acid (PGA). In one embodiment, the polymer may be nylon or polypropylene.

In one embodiment, at least one of the first anchoring element and the second anchoring element may include a T-bar element. At least one of the first anchoring element and the second anchoring element may include a material selected from the group consisting of a polymer, a metal, a plastic, a fabric, and combinations thereof. The metal may, for example, include stainless steel, aluminum, titanium, nickel-titanium, cobalt-chromium, and/or platinum. In one embodiment, at least one of the first anchoring element and the second anchoring element may include a hole, with the elongate suture wire extending through at least a portion of the hole.

Another aspect of the invention may include a suture deployment device. The suture deployment device may include a housing and a substantially hollow elongate needle including a slot extending along a length thereof. The substantially hollow elongate needle may be coupled at a proximal end to the housing. The distal end of the substantially hollow elongate needle may include a sharpened tip for piercing tissue. The suture deployment device may include a suture storage cartridge coupled to the housing and adapted to load a suture anchoring element into the proximal end of the substantially hollow elongate needle and a push rod adapted to retractably extend through the substantially hollow elongate needle and eject a loaded suture from a distal end of the substantially hollow elongate needle when extended. The suture deployment device may also include a deployment mechanism for retractably extending the push rod though the substantially hollow elongate needle.

In one embodiment, the substantially hollow elongate needle may include a reinforced proximal portion. The substantially hollow elongate needle may include a curvature along a longitudinal extent thereof. The slot may be adapted to provide a channel for passing at least a portion of an elongate suture wire during ejection of the suture. In one embodiment, the suture storage cartridge is releasably coupled to the housing. In one embodiment, the substantially hollow elongate needle and the suture storage cartridge are coupled together, and are jointly releasably coupled to the housing.

In one embodiment, the suture storage cartridge includes a spring mechanism. The suture storage cartridge may be adapted to load a suture anchoring element into the proximal end of the substantially hollow elongate needle when the push rod is retracted into the housing. The suture storage cartridge may be adapted to hold a plurality of suture anchoring elements. In one embodiment, the suture storage cartridge is loaded through the substantially hollow elongate needle. In one embodiment, the suture storage cartridge includes a preloaded, disposable cartridge.

In one embodiment, the deployment mechanism includes a triggering mechanism. The triggering mechanism may include at least one of a spring mechanism, a gear mechanism, a lever mechanism, a magnetic mechanism, a hydraulic mechanism, and an electrical mechanism. The electrical mechanism may, for example, include a motor. In one embodiment, the triggering mechanism is manually actuated. In additional embodiments, the device may include one or more optical fibers having a distal end(s) located adjacent the distal end of the needle to allow for visualization of the treatment area, for example, where visual access to the treatment site is otherwise compromised. Alternatively, the device may include clips or other mechanical fasteners for attaching the optical fiber(s) to the device. The clips could be disposed at various locations along the device housing and/or needle as necessary.

Another aspect of the invention may include a method of deploying a suture. The method may include the steps of providing a suture comprising a first anchoring element and a second anchoring element, with an elongate suture wire extending therebetween, and providing a suture deployment device including a substantially hollow elongate needle. The methods may also include deploying the first anchoring element into a first location of a treatment site of a patient through the substantially hollow elongate needle, deploying the second anchoring element into a second location of the treatment site through the substantially hollow elongate needle, cutting the elongate suture wire extending between the first anchoring element and a second anchoring element to create two elongate suture wire portions, and tying the two elongate suture wire portions together to anchor the first anchoring element and a second anchoring element.

In one embodiment, the method further includes loading the suture into the suture deployment device prior to deployment. The suture deployment device may include a triggering mechanism. The step of deploying the first anchoring element may include inserting a distal end of the substantially hollow elongate needle into the treatment site at a first location and actuating the triggering mechanism to eject the first anchoring element from the distal end of the substantially hollow elongate needle. The step of deploying the second anchoring element may include removing the distal end of the substantially hollow elongate needle from the treatment site, inserting the distal end of the substantially hollow elongate needle into the treatment site at a second location, and actuating the triggering mechanism to eject the second anchoring element from the distal end of the substantially hollow elongate needle.

The treatment site may include a hernia or a region of pelvic prolapse. The method may further include the step of placing a support element within the treatment site. The support element may, for example, include a patch for an urethrovesical suspension or ventral hernia procedure. At least one of the first anchoring element and second anchoring element may be deployed along a circumference of the support element.

These and other objects, along with advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a schematic side view of a suture with two anchoring elements, in accordance with one embodiment of the invention;

FIG. 1B is a schematic side view of another suture with two anchoring elements, in accordance with one embodiment of the invention;

FIG. 1C is a schematic side view of a suture with a barbed anchoring element joining two separated sections of a body part, in accordance with one embodiment of the invention;

FIG. 1D is a schematic side view of a suture with a plurality of anchoring elements deployed in a treatment site, in accordance with one embodiment of the invention;

FIG. 2A is a schematic end view of an anchoring element for a suture, in accordance with one embodiment of the invention;

FIG. 2B is a schematic side view of the anchoring element of FIG. 2A;

FIG. 2C is a schematic sectional view of the anchoring element of FIG. 2A, through the line C-C of FIG. 2A, with a suture inserted;

FIG. 3A is a schematic end view of an anchoring element for a suture, in accordance with one embodiment of the invention;

FIG. 3B is a schematic side view of the anchoring element of FIG. 3A;

FIG. 3C is a schematic sectional view of the anchoring element of FIG. 3A, through the line C-C of FIG. 3A, with a suture inserted;

FIG. 4A is a schematic end view of an anchoring element for a suture, in accordance with another alternative embodiment of the invention;

FIG. 4B is a schematic side view of the anchoring element of FIG. 4A;

FIG. 4C is a schematic sectional view of the anchoring element of FIG. 4A, through the line C-C of FIG. 4A, with a suture inserted;

FIG. 5A is a schematic end view of an anchoring element for a suture, in accordance with another alternative embodiment of the invention;

FIG. 5B is a schematic side view of the anchoring element of FIG. 5A;

FIG. 5C is a schematic sectional view of the anchoring element of FIG. 5A, through the line C-C of FIG. 5A, with a suture inserted;

FIG. 6A is a schematic side view of a substantially hollow needle for a suture deployment device, in accordance with one embodiment of the invention;

FIG. 6B is a schematic top view of the hollow needle of FIG. 6A;

FIG. 6C is a schematic side view of another substantially hollow needle for a suture deployment device, in accordance with one embodiment of the invention;

FIG. 6D is a schematic sectional top view of the substantially hollow needle of FIG. 6C through section A-A;

FIG. 7 is a schematic sectional side view of a hollow needle and suture loading element, with a suture loaded therein, in accordance with one embodiment of the invention;

FIG. 8A is a schematic side view of a suture deployment device, in accordance with one embodiment of the invention;

FIG. 8B is a schematic end view of the suture deployment device of FIG. 8A, with a suture loaded therein;

FIG. 8C is a schematic side view of the suture deployment device of FIG. 8A, with a suture loaded therein;

FIG. 8D is a schematic sectional view of the suture deployment device of FIG. 8A through the line D-D of FIG. 8B, prior to deployment of a suture anchoring element;

FIG. 8E is a schematic sectional view of the suture deployment device of FIG. 8A through the line D-D of FIG. 8B, during deployment of a suture anchoring element;

FIG. 8F is a schematic sectional view of the suture deployment device of FIG. 8A through the line D-D of FIG. 8B, with a plurality of sutures loaded therein;

FIGS. 9A-9I show a method of deploying a suture using a suture deployment device, in accordance with one embodiment of the invention;

FIG. 10 is a schematic sectional side view of a suture deployment device, in accordance with another alternative embodiment of the invention;

FIG. 11 is a schematic sectional side view of a suture deployment device, in accordance with another alternative embodiment of the invention;

FIG. 12 is a schematic sectional side view of a suture deployment device, in accordance with another alternative embodiment of the invention;

FIG. 13 is a schematic sectional side view of a suture deployment device, in accordance with another alternative embodiment of the invention; and

FIG. 14 is a schematic sectional side view of a suture deployment device, in accordance with another alternative embodiment of the invention.

### DESCRIPTION

In general, the present invention relates to methods and devices of deploying medical sutures, for example in urethrovesical suspension procedures, or other appropriate surgical techniques involving the deployment of sutures with anchoring elements.

For example, suture deployment devices have many potential uses in the placement of a suburethral sling, which vary according to where the suture anchoring elements are anchored. Once a suture anchoring element is deployed, the attached suture or mesh may be used to create a sling or hammock support underneath the urethra. An example procedure for anchoring a support, describing the placement of an anchoring element in the traditional position above the rectus fascia, is described in U.S. Pat. No. 6,595,911, the disclosure of which is hereby incorporated herein by reference in its entirety. Suture deployment devices may also be similarly used in the surgical repair of a hernia.

In one embodiment of the invention, the methods and devices described herein may be used to form a mini-sling where the suture anchoring elements are guided on either side of the bladder neck upwards but stopped prior to penetrating the rectus fascia. The suture anchoring elements may then be deployed in the fatty fibrous tissue, which lies beneath the rectus fascia but can still provide support. This would allow greater safety with reduced probability of bladder injury, bleeding, or nerve damage.

Another embodiment of the invention may include a procedure for the preparation of a pre-pubic sling, where the toggle device is inserted into the vagina, but rather than going behind the pubic bone goes in front of the pubic bone. The suture anchoring elements may then be deployed into the muscle tissue just underneath the skin. Again, the suture anchoring elements may be attached to a mesh or a suture, which could then be used to anchor any type of tissue underneath the bladder neck creating a sling/hammock. The suture deployment device may allow the procedure to be performed through smaller incisions, which potentially may allow this procedure to be performed in a medical office setting, rather than in a surgical theater. The pre-pubic approach has the advantage of not passing the needle through the retropubic space (behind the pubic bone), which could then risk bladder, bowel, or blood vessel injury.

Another embodiment of the invention may include a procedure for hernia repair or pelvic prolapse repair using a vault suspension, where the suture deployment device may be used to deploy sutures in a variety of structures lying high up in the pelvic region. Once the suture anchoring elements are placed, the attached suture may be used to anchor soft tissue or mesh/biologic graft for prolapse repair. The anchor may be deployed in a variety of support structures including the sacrospinous ligament, coccygeus muscle or arcus tendineus facia; all strong support structures that create high suspension of the vagina.

One example method of using the invention may include performing prolapse repair procedures without the need for a vaginal incision. As the suspension structures may be felt through the vagina, an examination may be performed, the suspension structures palpated, and the suture anchoring elements deployed through the vaginal wall without the need for a surgical incision. The resulting suture could then be tied down or tacked down, resulting in the creation of the required support. Subsequent scarring would reinforce the repair.

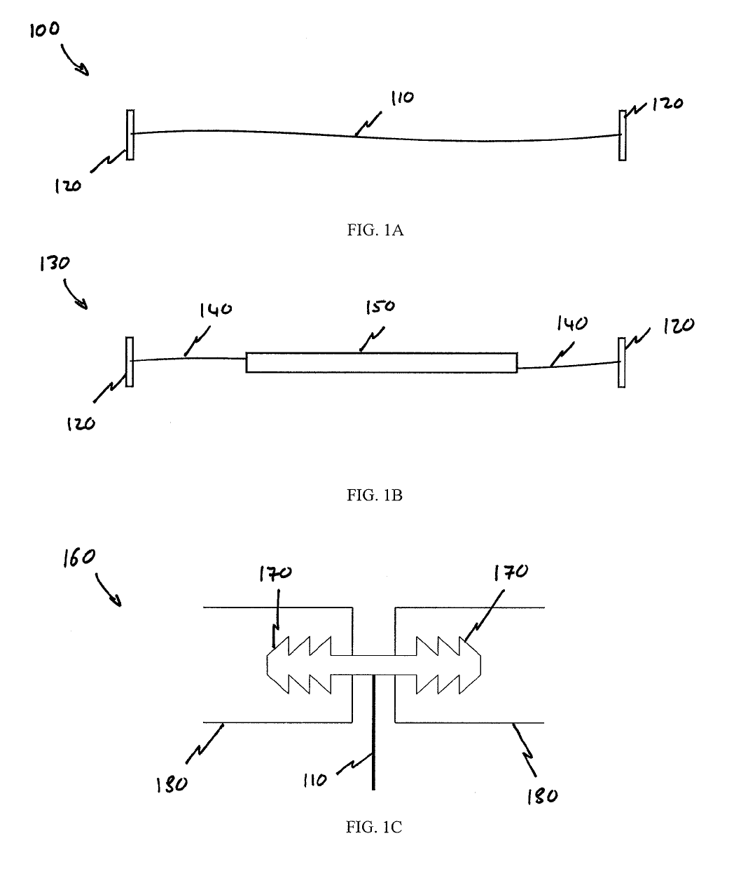

One embodiment of the invention includes a medical suture with an anchoring element, such as, but not limited to, a T-bar, at each end. An example suture 100 is shown in FIG. 1A. The suture 100 includes a suture wire 110 with an anchoring element 120 attached at each end.

The suture wire 110 may be of any appropriate length necessary to carry out the specific medical procedure of interest. In one example embodiment, the suture wire 110 may range from approximately 50 mm to 300 mm, and more particularly from 100 mm to 200 mm. The suture wire 110 may, in one embodiment, have a diameter ranging from 0.3 mm to 3 mm, as appropriate. In one embodiment, the suture wire 110 may include distal portions of a first diameter, with a central portion of a different diameter. In one embodiment, as shown in FIG. 1B, a suture 150 includes two anchoring elements 120, with a suture wire 130 including thin end portions 140, with a thicker central portion 150. This thicker central portion may, for example, be advantageous in easing the manufacture of a suture wire, such as a suture wire manufactured using an injection molding technique.

The suture wire 110 and/or anchoring elements 120 may be manufactured from a material including a metal, a plastic, a polymer, a textile, a composite material, or combinations thereof. For example, in one embodiment, the suture wire 110 and anchoring elements 120 are both constructed from an absorbable material, such as, but not limited to, an absorbable polylactic acid (PLA), polyglycolic acid (PGA), or combinations thereof. In another embodiment, at least one of the suture wire 110 and/or anchoring elements 120 may be constructed from a permanent material such as, but not limited to, polypropylene, titanium, aluminum, or stainless steel.

The suture wire 110 and anchoring elements 120 may be constructed from the same material, or from different materials. In one embodiment, both the suture wire 110 and anchoring elements 120 are constructed as a one piece molded unit from a resorbable material. In an alternative embodiment, the suture 110 may be constructed as a two, or more, piece insert molded structure. This would allow for two different hardness materials to be used for suture wire 110 and anchoring elements 120.

In one embodiment, the suture 100 may include two anchoring element 120, one at each end of the suture wire 110. In an alternative embodiment, additional anchoring elements 120 may be placed along the length of the suture wire 110, thereby allowing a single suture 100 to be anchored at more than two locations. In a further alternative embodiment, the suture 100 may only have an anchoring element 120 at one end, or have differently shaped and/or configured anchoring elements 120 at each end. In one embodiment, the anchoring elements 120 may be slightly curved and/or flexible in order to allow them to pass through a curved needle of a deployment device.

The anchoring elements 120 at each end of the suture wire 110 may be of the same size and material, or different sized and materials, as appropriate. In one example, the anchoring elements may have a diameter ranging from approximately 1 mm to 3 mm, and an elongate length ranging from approximately 5 mm to 25 mm.

In an alternative embodiment, one or more anchoring elements may include a barbed portion. An example barbed anchoring element 160, including barbed end portions 170, is shown in FIG. 1C. The barbed end portions 170 may, for example, allow the anchoring element 160 to be placed between two separated sections of a body part 180, such as, but not limited to, a bone, a tendon, or other appropriate body part, and provide an anchoring means to rejoin these separated sections 180.

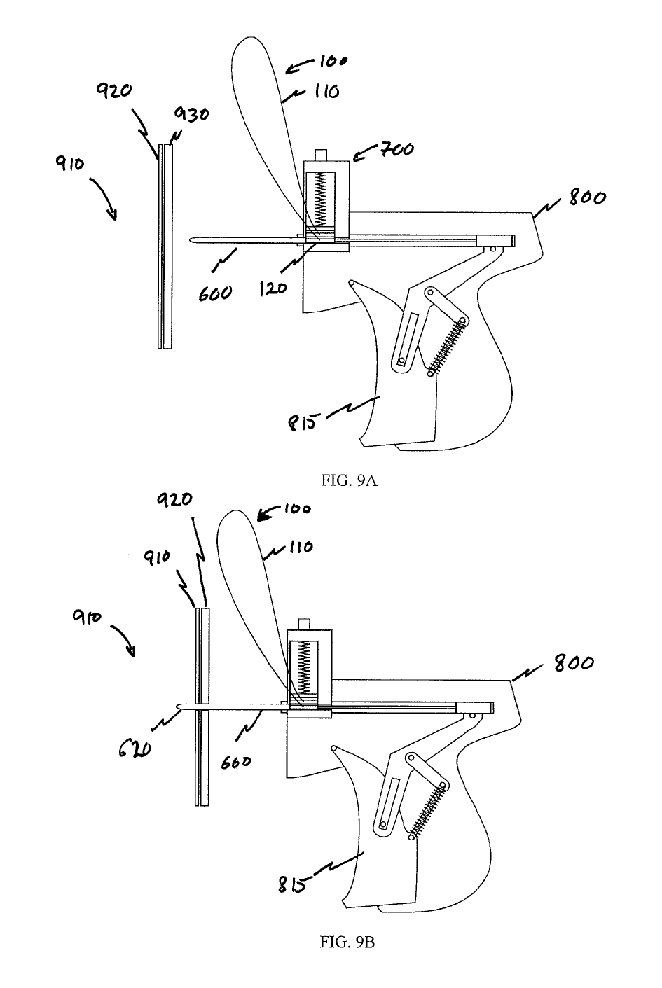

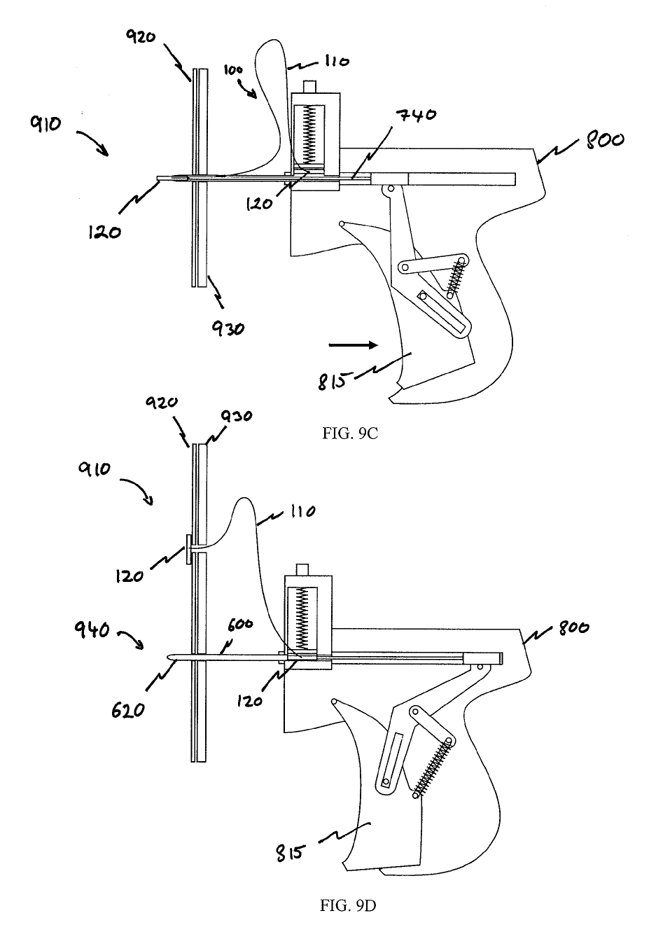

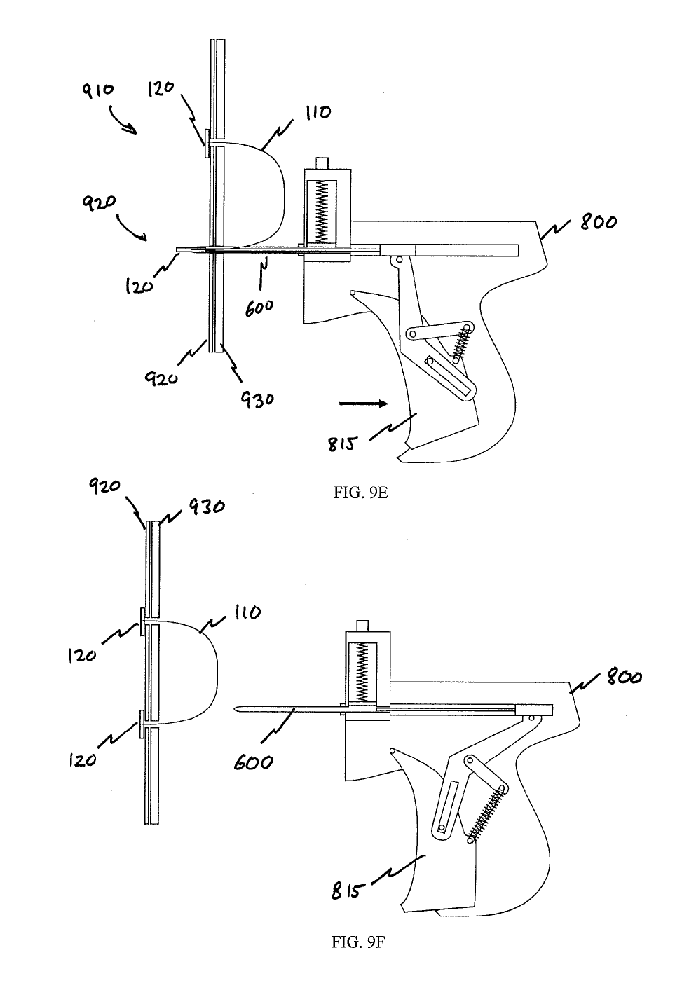

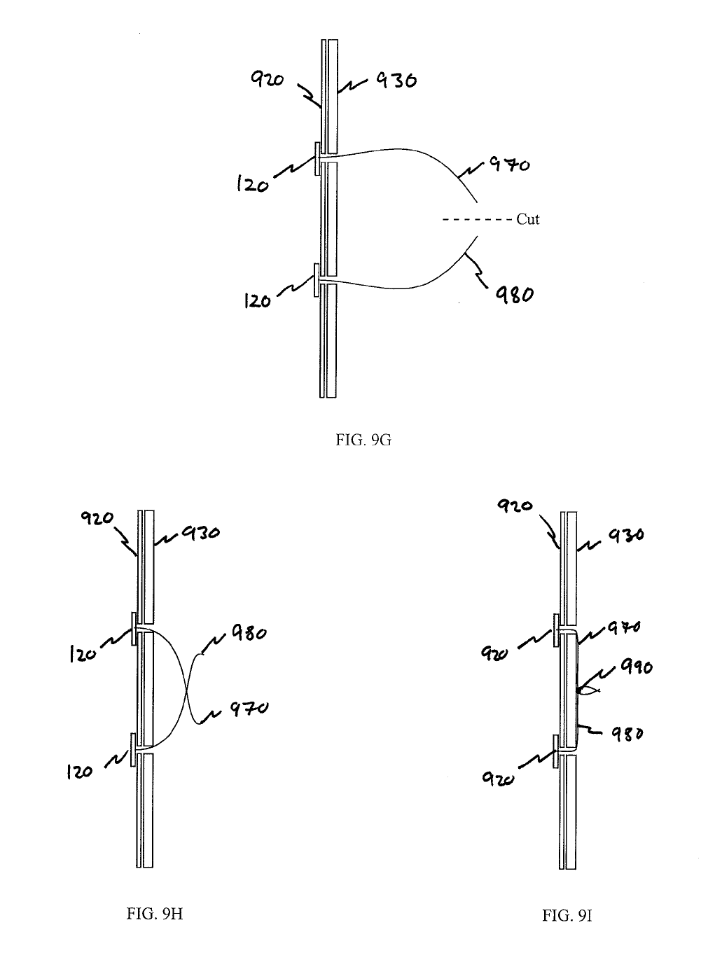

In an another embodiment, a suture wire 110 may be connected to a plurality of anchoring elements 120, with, for example, an anchoring element 120 located at each end of the suture wire 110, and additional anchoring elements 125 positioned at discrete intervals along the length of the suture wire 110. An example suture 190 including a plurality of anchoring elements 125 positioned along the length of the suture wire 110, is shown in FIG. 1D. The suture 190 is shown with the anchoring elements 120, 125 deployed within a treatment site and inserted through tissue 192 and a support element 194. A method of deploying a suture is described below for FIGS. 9A to 9I. The suture 190 may include any number of anchoring elements 125, as required.

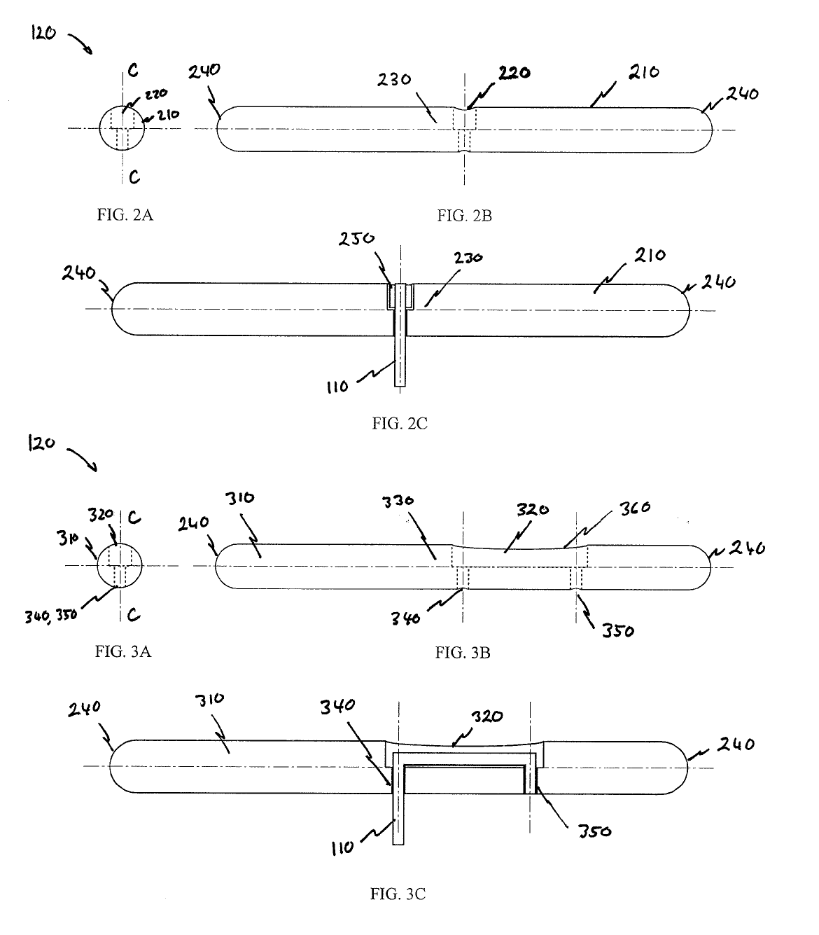

The anchoring elements 120 may be attached to the suture wire 110 by any appropriate means, such as, but not limited to, a mechanical connection, such as crimping or tying, or by bonding. Example anchoring elements are shown in FIGS. 2A-5C.

The anchoring element 120 shown in FIGS. 2A-2C includes a solid elongate shaft 210 with a hole 220 drilled through a central portion 230 to provide an attachment location for the suture wire 110. To secure the anchoring element 120 to a suture wire 110, a distal end of the suture wire 110 may be passed through the hole 220 and crimped, thereby forming a thicker end portion of the suture wire 110 that may be securely fitted within the hole 220. In one embodiment, a separate crimping element 250 may be crimped onto the suture wire 110. The separate crimping element may, for example, include a ring, a ball, a cylinder, a sleeve, a sheet or other appropriate element, and may be manufactured from a material including, but not limited to, a metal (such as stainless steel or aluminum), a plastic, and/or a fabric. The crimping element may be of any appropriate length necessary to provide a sufficient bond between the suture wire 110 and anchoring element 120.

In an alternative embodiment, the suture wire 110 may be folded over at its distal end and crimped to itself. In one embodiment, the crimped wire portion may be bonded to at least a portion of the walls of the hole 220 to further secure the anchoring element 120 to the suture wire 110. In an alternative embodiment, the crimped portion may be secured within the hole 220 through a simple pressure fitting.

In one embodiment, the anchoring element 120 may include rounded distal ends 240 in order to ensure that the anchoring element 120 is atraumatic (i.e. to prevent the ends from cutting or otherwise harming any tissue or other body substance that it touches).

Alternative configurations of anchoring element 120 are shown in FIGS. 3A-5C. The anchoring element 120 shown in FIGS. 3A-3C includes a solid elongate shaft 310 with a cutout section 320 extending along a central portion 330 of the solid elongate shaft 310. A first hole 340 extends through the central portion 330 of the anchoring element 120 and into the cutout section 320, with a second hole 350 extending back through the solid elongate shaft 310 at a distal end 360 of the cutout section 320. In an alternative embodiment, the second hole 350 is not included, with only the first hole 340 extending into the cutout section 320. The suture wire 110 may be bonded or otherwise attached to the anchoring element 120 through the holes 340, 350 and cutout section 320, as appropriate. The suture wire 110 may be bonded to the anchoring element 120 by any appropriate bonding, such as, but not limited to, adhesive bonding, welding, and/or heat bonding.

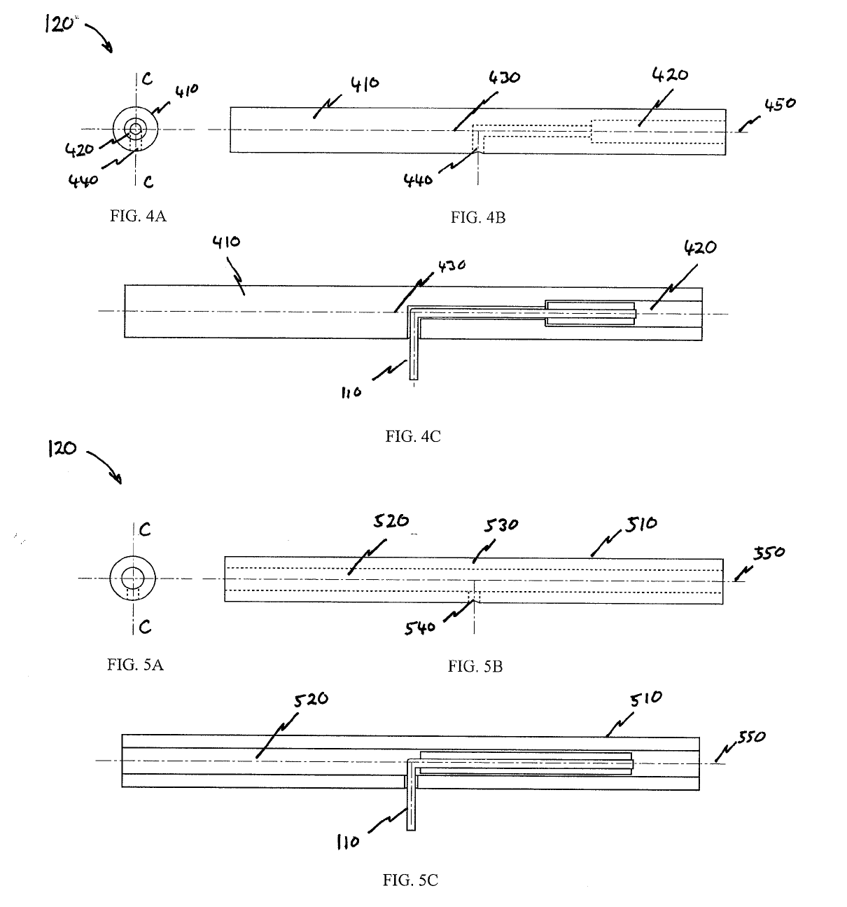

The anchoring element 120 shown in FIGS. 4A-4C includes a solid elongate shaft 410 with a elongate hole 420 extending along a central axis 450 of the solid elongate shaft 410. A hole 440 extends through the central portion 430 of the anchoring element 120 and into the elongate hole 420, thereby providing a location for the attachment of a distal end of the suture wire 110 through crimping, bonding, and/or any other appropriate means. The elongate hole 420 may be a stepped hole, thereby providing an abutment location for the crimped portion to rest against, and preventing slippage of the suture wire 110.

The anchoring element 120 shown in FIGS. 5A-5C includes a hollow elongate shaft 510 with a hollow interior 520 extending along a central axis 550 of the solid elongate shaft 510. A hole 540 extends through the central portion 530 of the anchoring element 120 and into the hollow interior 520, thereby providing a location for the attachment of a distal end of the suture wire 110 through crimping, bonding, and/or any other appropriate means. In a further alternative embodiment and other appropriate means of fixedly or removably attaching the anchoring elements 120 to the distal ends of the suture wire 110 may be used, as appropriate.

In operation, the suture 100 may be inserted into a target location of a body to provide any one of a number of medical functions. For example, the suture 100 may be used to close a wound or surgical incision in a patient, or secure an internally and/or externally placed support element, or other medical treatment element or device. In one embodiment, one or more sutures 100 are used to anchor a support element, such as, but not limited to, an urethrovesical suspension procedure. The anchoring elements may be deployed around at least a portion of a circumference of the support element.

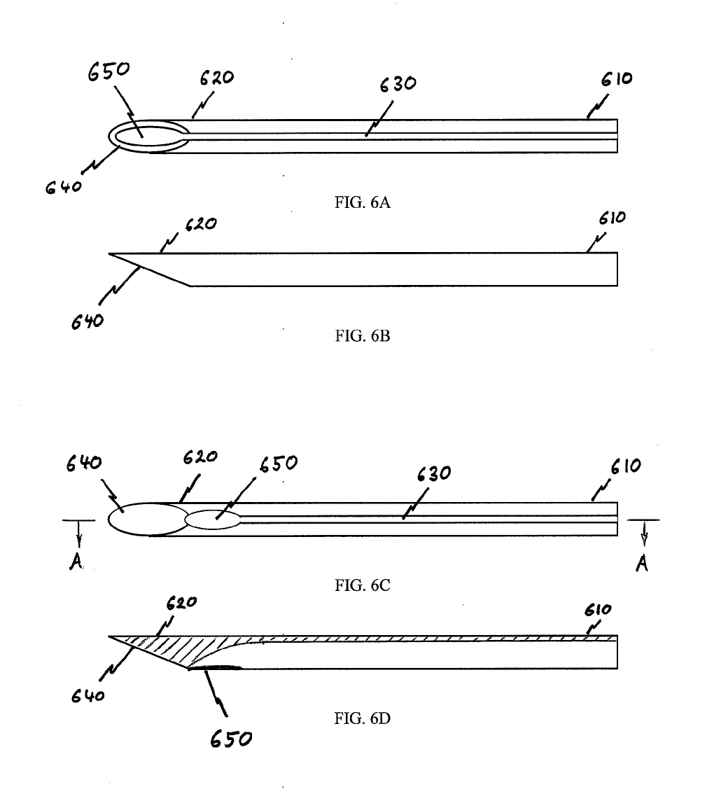

In operation, the anchoring elements 120 of a suture 100 may be deployed through a substantially hollow elongate needle and into a treatment location of a patient. An example hollow elongate needle 600 for use in deploying a suture 100 is shown in FIGS. 6A-6B. The needle 600 includes a proximal end 610, for attaching to a housing of a suture deployment device, and a distal end 620 for insertion into a treatment site of a patient. The needle may, in one embodiment, include a slot 630, such that while an anchoring element 120 is passed through the hollow central extent of the needle 600, the suture wire 110 is free to extend out through the slot 630 and thereby be free of the needle upon deployment of the anchoring elements 120. This allows a suture 100 with anchoring elements 120 at each end to be deployed easily without the suture wire 110 remaining within the needle 600 during deployment of the second anchoring element 120 and thereby blocking the deployment of the second anchoring element 120.

In one embodiment, the distal end of the needle 600 includes an angled tip portion 640, which can be used to pierce the boundary of the treatment site, such as, but not limited to, the skin or other body portion of the patient, to allow the needle to be extended into the treatment site. The anchoring elements may then exit the needle 600 through a deployment port 650 in the tip portion 640. In one embodiment, the needle 600 may include a reinforced section to strengthen the needle 600 during piercing of the treatment site boundary. This reinforced section may include a region of thicker needle material, a reinforcing sleeve, or another appropriate strengthening means. The needle 600 may, in one embodiment, be reinforced at a distal end, a proximal end, and/or a central portion. The angle of the tip portion 640 may be set at any appropriate angle to allow it to safely and easily pierce the boundary of the treatment site, and may be sharper of blunter than the angle shown in FIGS. 6A and 6B, as appropriate.

In one embodiment, the needle may be of any appropriate dimensions necessary to carry appropriately sized suture anchoring elements 120 and extend into appropriate body regions. For example, in one embodiment, the needle 600 may have an outer diameter ranging from approximately 1-3 mm, and a length ranging from approximately 20-100 mm. In one embodiment, the needle 600 may have a consistent wall thickness along a length thereof. In an alternative embodiment, the wall thickness of the needle may vary along the length of the needle 600. In one embodiment, the inner and/or outer diameter of the needle 600 may vary along a length thereof.

In one embodiment, the needle 600 may be curved along at least a portion of its longitudinal extent. As a result, the needle 600 may be inserted into the treatment site of a patient along a curved trajectory, thereby allowing an anchoring element 120 to be inserted at an angle and/or allowing the needle 600 to avoid an obstruction when entering the treatment location. In one embodiment, the hollow interior surface of the needle 600 may include a low friction material to ease the travel of the anchoring elements 120 and/or push rod.

In one embodiment, the needle 600 includes a substantially solid tip portion 640, with a deployment port 650 located at the distal end 620 of the needle 600 behind the tip portion 640. An example needle 600 including a solid tip portion 640 is shown in FIGS. 4C and 4D. In operation, an anchoring element 120 may be passed through the hollow central extent of the needle 600 with the suture wire 110 free to extend out through the slot 630, as described above. Upon reaching the distal end 620, the anchoring element 120 passes from the needle 600 through the deployment port 650 and into a treatment site. In alternative embodiments, the deployment port 650 may be located at any position along the elongate length of the needle 600.

Providing a solid tip portion 640 with a deployment port 650 extending out of a side of the needle 600 behind the tip portion 640 may, in some embodiments, reinforce the tip portion 640 and/or reduce the trauma applied to the tissue within the treatment site during insertion of the distal end 620 into the treatment site. For example, by placing the deployment port 650 on the side of the needle 600 behind a solid tip portion 640, the edges of the deployment port 650 do not impinge directly on the tissue through which the needle 600 is passed, and therefore do not apply additional trauma to the tissue beyond that applied by the tip portion 640 itself. The tip portion 640 may, in certain embodiments, include curved edges, or other shaped elements, to further reduce the trauma on the tissue.

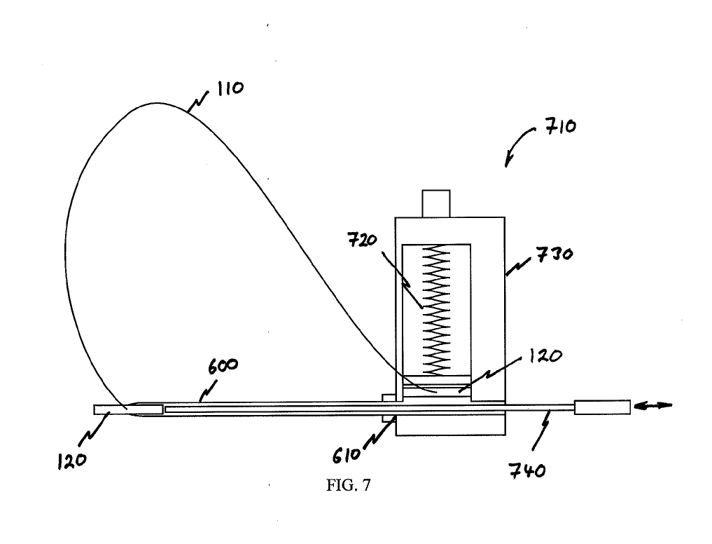

In one embodiment, the needle 600 may be mounted at its proximal end 610 to a suture storage cartridge 700. A sectional side view of the example needle 600 and suture storage cartridge 700 through the central axis of the needle can be seen in FIG. 7. The suture storage cartridge 700 may be used to store anchoring elements 120 for one or more sutures 100 in a preferred orientation, and load the anchoring elements 120 sequentially and one at a time into the needle 600 ready for deployment.

In one embodiment, the suture storage cartridge 700 includes a spring-loaded mechanism 710, including a spring 720, housed within a storage cartridge housing 730. The storage cartridge housing 730 is adapted to hold a plurality of suture anchoring elements 120 such that the spring 720 preferentially forces a single anchoring element 120 into the proximal end 610 of the needle 600 when it is empty. In an alternative embodiment, a ratchet mechanism, a screw mechanism, a motor driven mechanism, a pneumatic mechanism, or any other appropriate mechanical, electronic, and/or magnetic mechanism may be used to preferentially force anchoring elements 120 into the proximal end 610 of the needle 600 when it is empty.

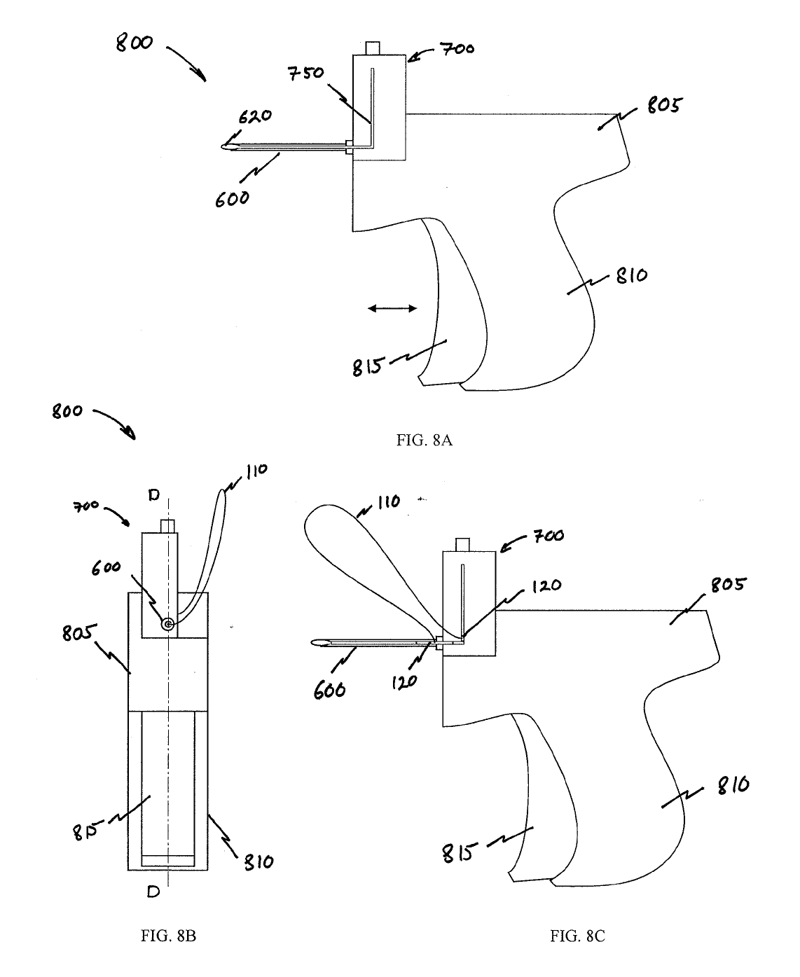

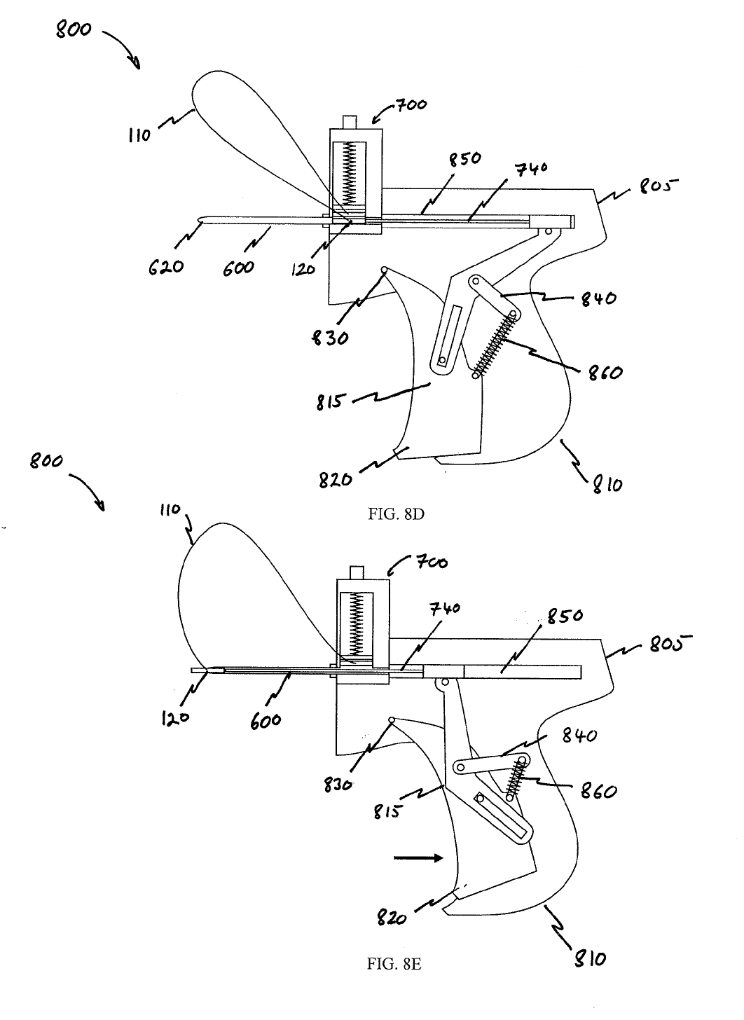

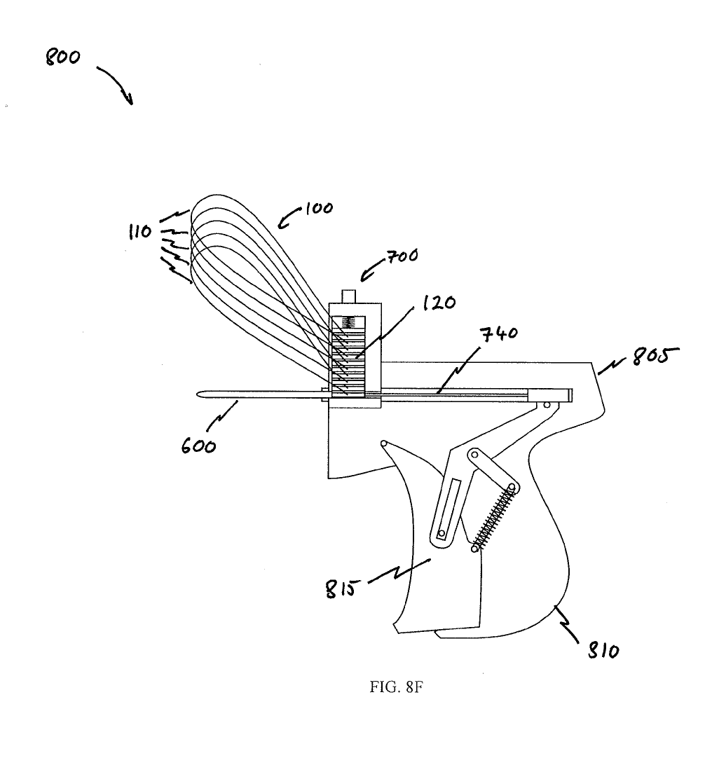

The storage cartridge housing 730 may be configured to hold any number of suture anchoring elements, as required. The storage cartridge housing 730 may include a slot 750 (shown in FIGS. 8A and 8C) in communication with the needle slot 630, allowing the suture wires 110 to extend clear of the device and thereby avoid blocking the deployment of the anchoring elements 120.

In one embodiment, the storage cartridge housing 730 and needle 600 may be permanently attached to a housing of a suture deployment mechanism for deploying the anchoring elements 120. In an alternative embodiment, the storage cartridge housing 730 and/or needle 600 may be modularly designed so that it may be removed from the housing of the suture deployment mechanism and replaced with newly loaded cartridge 730 and fresh sharp needle 600.

In one embodiment, anchoring elements 120 may be loaded into the storage cartridge housing 730 through the hollow elongate needle 600. In an alternative embodiment, anchoring elements 120 may be loaded into the storage cartridge housing 730 through a separate loading passageway within the storage cartridge housing 730. In a further alternative embodiment, the storage cartridge housing 730 may be loaded with anchoring elements 120 prior to being coupled to the needle 600 and/or deployment device.

In one embodiment, the anchoring elements 120 may be deployed by a push rod 740. This push rod 740 may be extended and retracted in a reciprocating motion. For example, at rest, the push rod 740 may be positioned in a retracted position. The multiple anchoring elements 120 for the sutures 100 may be loaded, one at a time, into the needle 600 by the spring-loaded mechanism 710. In this embodiment a suture deployment device (i.e. a suture tagging device) may hold multiple sutures 100 with pairs of anchoring elements 120 attached thereto. In one embodiment, when the push rod 740 is extended into the needle 600, it blocks the suture storage cartridge 700 and prevents additional anchoring elements 120 from being inserted into the needle 600. However, if the push rod 740 is retracted out of the needle to its fully retracted position, it moves clear of the suture storage cartridge 700, thereby allowing a new anchoring element 120 to be inserted into the needle 600. The push rod 740 may be manufactured from a metal, a plastic, or any other appropriate material. In one embodiment, the push rod 740 is manufactured from nickel-titanium (Nitinol™).

In operation, the suture deployment device may deploy a suture anchoring element 120 by actuating the push rod 740 to extend through the hollow elongate needle 600. This actuation may be achieved through a mechanical actuation mechanism, an electrical actuation mechanism, a magnetic actuation mechanism, a hydraulic actuation mechanism, or a combination thereof. The actuation mechanism may be manually actuated and/or be automatically actuated in response to an instruction from a control device.

In one example embodiment, the actuation mechanism is a mechanical trigger mechanism. This trigger mechanism advances the distal end of push rod 740 through the hollow elongate needle 600, thereby pushing a single anchoring element 120 through and out of the distal end of the needle 600. The push rod 740 pushes the first anchoring element 120 from out of the needle 600 and into the treatment site. When pressure is released from the trigger mechanism, the push rod 740 returns to its original retracted position, allowing another suture anchoring element 120 to be loaded into the hollow elongate needle 600 and thereby preparing the device for deployment of another suture anchoring element 120.

An example suture deployment device including a triggering mechanism is shown in FIGS. 8A-8F. In this embodiment, the suture deployment device 800 includes a housing 805 including a handle element 810, with a triggering mechanism 815 held therein. A suture storage cartridge 700 and a needle 600 are mounted to the housing 805 such that in operation, actuation of the triggering mechanism results in the deployment of a suture anchoring element 120 from the distal end 620 of the needle 600.

In one embodiment, the triggering mechanism 815 includes a pivoting mechanism adapted to extend and retract a push rod 740 through the needle 600 upon actuation. In this embodiment, the triggering mechanism 815 includes a user interface element 820 (in this case a trigger) that pivots about a pivot point 830. A pivoting lever mechanism 840 is coupled to the user interface element 820 and the push rod 740 such that, upon a pivoting of the user interface element 820 about the pivot point 830, the push rod 740 is forced along a track 850 such that its distal end extends through the hollow elongate needle 600. When the push rod 740 is fully extended, the anchoring element 120 is pushed from the distal end 620 of the needle 600 and into the treatment site. A schematic sectional view of an example triggering mechanism 815 with the push rod 740 extended is shown in FIG. 8E.

In one embodiment, the pivoting lever mechanism 840 includes a spring element 860 that is adapted to provide a restoring force to the user interface element 820, thereby returning the triggering mechanism 815 to its “at rest” position where the push rod 740 is fully retracted into the housing 805. A schematic sectional view of an example triggering mechanism 815 with the push rod 740 retracted (i.e. in an “at rest” position) is shown in FIG. 8D. When fully retracted, the suture storage cartridge 700 is free to load another anchoring element 120 into the proximal end 610 of the needle 600, thereby placing the suture deployment device 800 in condition to deploy another anchoring element 120. An alternative embodiment of the pivoting lever mechanism 840 may not include a spring element, such that the triggering mechanism 815 must be returned manually to its “at rest” position by the user. A schematic sectional view of an example suture deployment device 800 with the a plurality of anchoring elements 120 for a plurality of sutures 100 loaded within the suture storage cartridge 700 is shown in FIG. 8F.

An example method of performing a hernia or prolapse repair procedure to secure a support element to a treatment site of a patient is shown in FIGS. 9A-9I. A support element 920 is first inserted into a first access location 910 of the treatment site and positioned against the relevant tissue 930. The suture deployment device 800 can then be positioned outside the body at the treatment site, with the triggering mechanism 815 in an “at rest” position, as shown in FIG. 9A. Once positioned, the needle 600 may be pushed through the tissue 930 and support element 920 so that the sharpened distal end 620 of needle 600 extends into the first access location 910 beyond the support element 920, as shown in FIG. 9B.

Once the distal end 620 of the needle 600 is positioned correctly, the triggering mechanism 815 may be actuated to force the push rod 740 through the needle 600, thereby pushing a first anchoring element 120 of a suture 100 out of the distal end 620 of the needle 600 and into the first access location 910, as shown in FIG. 9C. Once the first anchoring element 120 has been deployed, the needle 600 can be retracted from the first access location 910, leaving the anchoring element 120 in place against the support element 920, with the suture wire 110 extending through the support element 920 and tissue 930 and back to the second anchoring element 120 still stored within the suture deployment device 800. As the suture wire 110 is free to extend out from the suture deployment device 800 through the needle slot 630 and the suture storage cartridge slot 750, the suture wire 110 will not have to pass through the needle 600. The suture wire will, therefore, not block or affect the deployment of the second anchoring element 120.

Once the first anchoring element 120 is in position with the needle 600 removed, the needle 600 can be inserted through the tissue 930 and support element 920 and into a second access location 940 of the treatment site, as shown in FIG. 9D. The second anchoring element 120 can then be deployed through the needle 600 and into the second access location 940 by triggering of the triggering mechanism 815 of the suture deployment device 800, as shown in FIG. 9E.

Once both anchoring elements 120 are correctly positioned within the treatment site, with the suture wire 110 extending therebetween, the suture deployment device 800 may be removed, as shown in FIG. 9F. Once the suture deployment device 800 has been removed, the suture wire 110 may be cut at a central portion 960, as shown in FIG. 9G. The two separate suture wire section 970, 980 (each extending to an anchoring element 120 anchoring the support element 920 within the treatment site) can then be tightly tied together, as shown in FIGS. 9H and 9I, thereby anchoring the support element 920 firmly and tightly against the tissue 930. Once a knot 990 has been tied in the wire sections 970, 980, the free ends of the wire sections 970, 980 may be cut, thereby tidying up the treatment site. This procedure may be repeated at a plurality of locations around a circumferential edge of a support element 920 to anchor it firmly to the tissue 930 over its full extent and thereby provide support for the treatment site.

It should be noted that providing a suture 100 with anchoring elements 120 at each end of the suture wire 100 provides significant advantages. For example, because the suture wire 110 extends between the two anchoring elements 120 outside the body, a surgeon may insert a number of these sutures 100, according to the method of FIGS. 9A to 9I for example, without fear of losing a suture 100 within the body. More particularly, in one embodiment of the invention, the step of cutting and tying the suture wire 110 to anchor the suture 100 tightly against the support element 920 and relevant tissue 930 may be performed after anchoring elements 120 for a plurality of sutures 100 have been deployed, thereby making the deployment of these sutures 100, quicker, more efficient, and safer. In contrast, if a suture only had a single anchoring element, with the other end of the suture wire free, two sutures would have to be inserted and thereafter tied together. In that instance, the first suture would have to be held by a separate instrument while the second suture is being deployed, otherwise the first suture could slip through the access hole and into the treatment site while the second suture is being deployed. This may be of particular issue, for example, in hernia or prolapse repair procedures, where the tissue 930 may have to be manipulated rather significantly during the treatment procedure.

In one embodiment, the suture deployment device 800 may be loaded with multiple sutures 100 so that this procedure can be performed repeatedly without reloading the suture deployment device 800. As a result, a support element 910 can be anchored at numerous locations quickly and safely.

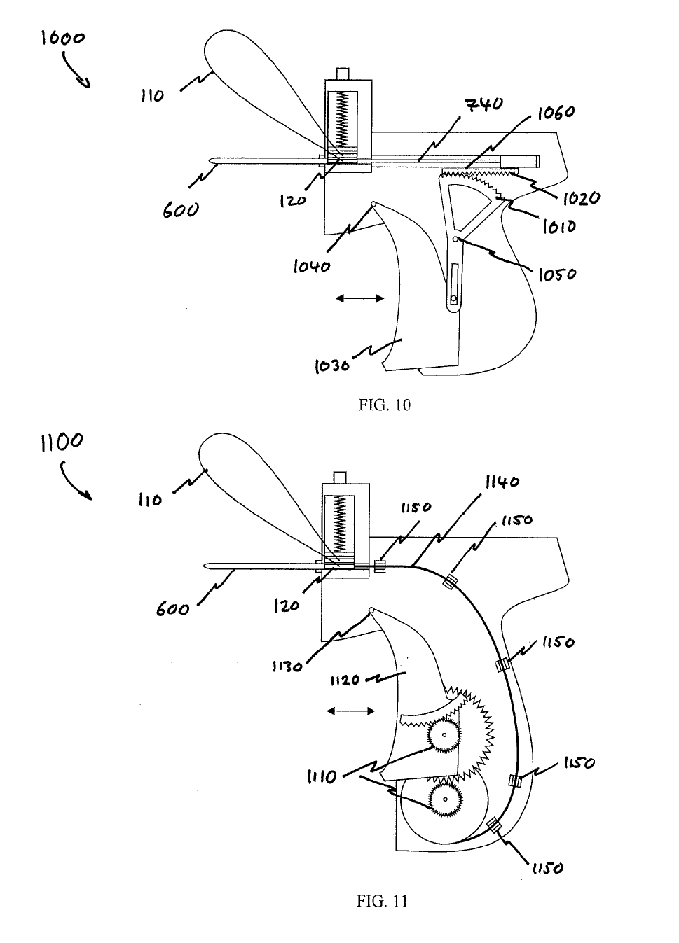

In various embodiments of the invention a push rod may be actuated by any appropriate mechanical, electronic, and/or magnetic mechanism. For example, a gear mechanism, such as, but not limited to, a rack and pinion gear mechanism, may be used to drive a flexible push rod. Example suture deployment devices including a gear mechanism and flexible shaft, in accordance with one embodiment of the invention, are shown in FIGS. 10-13.

In one embodiment of the invention, the pivoting lever mechanism 840 of a suture deployment device 1000 may be replaced by a gear section 1010 and toothed rack 1020, as shown in FIG. 10. In this embodiment, actuation of a pivoting user interface element 1030 about a first pivot point 1040 causes the gear section 1010 to pivot about a second pivot point 1050, thereby driving the toothed rack 1020 along a track 1060. As the toothed rack 1020 is attached to the push rod 740, actuating of the user interface element 1030 drives the push rod 740 through the needle 600, as before. In one embodiment, a spring element may be added to preferentially force the push rod 740 towards an “at rest” retracted position.

FIG. 11 shows a suture deployment device 1100 with a gear train mechanism 1110. In this embodiment, actuation of a pivoting user interface element 1120 about a first pivot point 1130 causes the gear train mechanism 1110 to push a flexible push rod 1140 through the needle 600, thereby deploying an anchoring element 120. The flexible push rod 1140 may pass through a number of guiding elements 1150 that are positioned to ensure a smooth extension of the flexible push rod 1140 into the needle 600. Again, a spring element may be added to preferentially force the push rod 1140 towards an “at rest” retracted position. The push rod 1140 may be constructed from a metal, a plastic, or any other appropriate material. In one embodiment, the push rod 740 is constructed from Nitinol™.

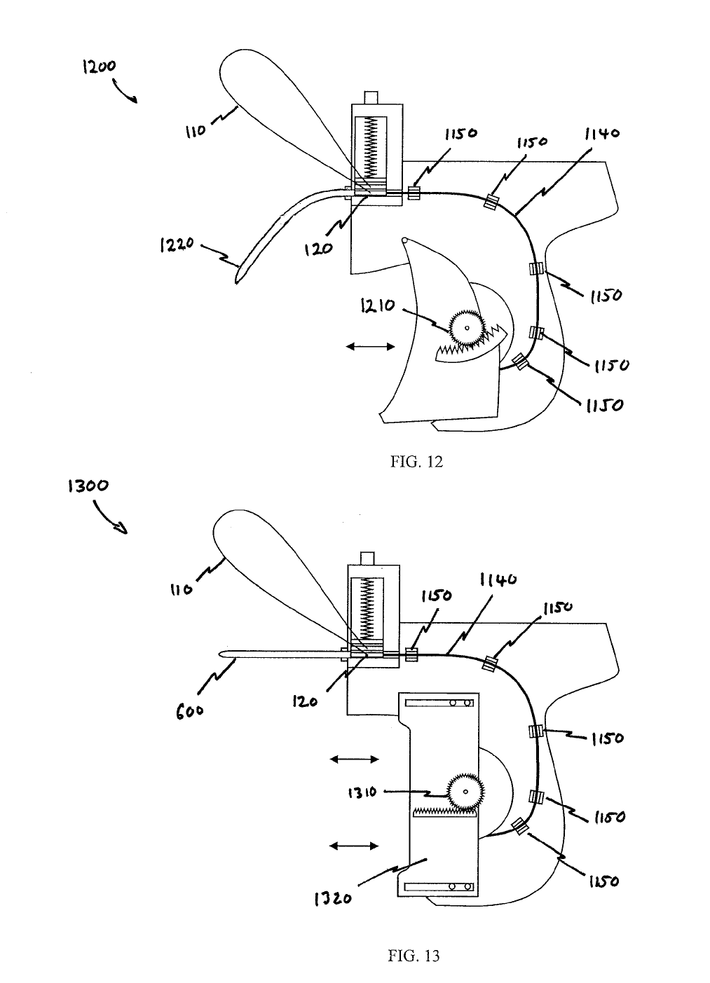

An alternative embodiment of the invention, with a suture deployment device 1200 including an alternative gear train mechanism 1210, is shown in FIG. 12. The suture deployment device 1200 also includes a curved hollow elongate needle 1220, allowing the anchoring element 120 to be deployed along a curved path, for example to avoid an obstruction when entering the treatment location. The needle 1220 may be curved in any direction, as appropriate. The angle of curvature of the needle 1220 may range from 0° to 90°. For example, in one embodiment, the angle of curvature of the needle may range from 30° to 75°. In operation, by utilizing a flexible push rod 1140, the anchoring elements 120 (which may themselves be flexible, curved, and/or treated with a low friction material to ease passage through the curved needle 1220) may be deployed through a wide range of curved needle geometries, as required.

In another alternative embodiment, the needle 1220 may include a curvature control element, thereby allowing the needle 1220 to be set to a required curvature for each deployment procedure and/or by guidably curved during insertion into a treatment site. In a further embodiment, the needle may include a flexible portion to facilitate the bending of the needle in a set direction, and/or to allow a user to adjustably set the needle at a specific angle prior to insertion into a treatment site, or guide the needle during insertion.

A further alternative embodiment of the invention, with a suture deployment device 1300 including an alternative gear train mechanism 1310 coupled to a linearly moving user interface element 1320, is shown in FIG. 13.

In alternative embodiments other appropriate gearing mechanisms may be used, including larger and/or smaller components, as appropriate. In various embodiments, one or more gears may be manufactured from a material including, but not limited to, a plastic, a metal, a composite material, and combinations thereof. The flexible push rod may be may constructed from a material that has sufficient strength and rigidity to maintain appropriate column strength to overcome spring pressures from a suture loading mechanism and drive an anchoring element 120 through the needle 600 and into a treatment site. For example, in one embodiment, the flexible push rod may be made of a plastic, a metal, a composite material, and combinations thereof. More particularly, in one example embodiment, the flexible push rod may be manufactured from a nylon coated stainless steel wound cable, a Nitinol™ coiled member, or any other appropriate material.

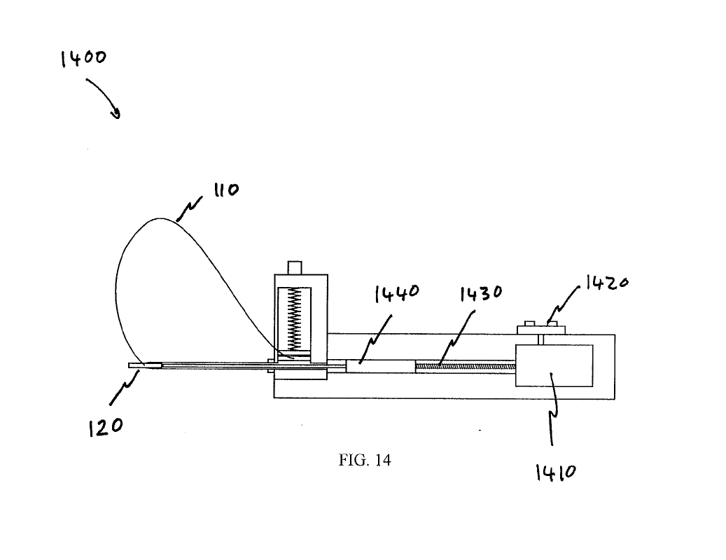

In one embodiment, the actuation of the push rod may be performed through a smooth reciprocation motion or through a step motion including a number of deployment stages or levels. In another embodiment, the push rod may be actuated by a stepper motor or micro-motor, such as, but not limited to, a Portescap™ 20DAM-L Digital Linear Actuator (which is a non-captive version of a micro-motor), available from Danaher Motion Portescap at 110 Westtown Road, West Chester, Pa. 19382. Utilizing a micro-motor allows for smaller and differently shaped suture deployment devices that do not, for example, require a pistol-type grip. An example suture deployment device including a micro-motor is shown in FIG. 14.

The embodiment of FIG. 14 may include a suture deployment device 1400 including a motor 1410 with a user control interface 1420. The user control interface 1420 may include one or more buttons, switches, toggles, sliding elements, touch screen elements, or any other appropriate electromagnetic motor control interface element. The motor 1410 may be connected through a drive shaft 1430 to a push rod 1440, such that actuation of the motor 1410 drives the push rod 1440 through the needle 600 in response to control signals from the user control interface 1420.

It should be noted that all of the devices described herein may be used to perform a surgical procedure, such as, but not limited to, a hernia or prolapse repair procedure, in accordance with the methods of FIGS. 9A-9I.

It should be understood that alternative embodiments, and/or materials used in the construction of embodiments, or alternative embodiments, are applicable to all other embodiments described herein.

The invention may be embodied in other specific forms without departing from the spirit or essential characteristics thereof. The foregoing embodiments, therefore, are to be considered in all respects illustrative rather than limiting the invention described herein. Scope of the invention is thus indicated by the appended claims, rather than by the foregoing description, and all changes that come within the meaning and range of equivalency of the claims are intended to be embraced therein.

What is claimed is:

1. A medical suture, comprising:
an elongate suture wire;
a first anchoring element connected to a first end of the elongate suture wire; and
a second anchoring element connected to a second end of the elongate suture wire.

2. The medical suture of claim 1, wherein the medical suture comprises a material selected from the group consisting of a polymer, a metal, a plastic, a fabric, and combinations thereof.

3. The medical suture of claim 2, wherein the polymer comprises an absorbable polymer.

4. The medical suture of claim 3, wherein the absorbable polymer comprises at least one of absorbable polylactic acid and absorbable polyglycolic acid.

5. The medical suture of claim 2, wherein the polymer comprises at least one of nylon and polypropylene.

6. The medical suture of claim 1, wherein at least one of the first anchoring element and the second anchoring element comprises a T-bar element.

7. The medical suture of claim 1, wherein at least one of the first anchoring element and the second anchoring element comprises a material selected from the group consisting of a polymer, a metal, a plastic, a fabric, and combinations thereof.

8. The medical suture of claim 7, wherein the metal is selected from the group consisting of stainless steel, aluminum, titanium, nickel-titanium, cobalt-chromium, and platinum.

9. The medical suture of claim 1, wherein at least one of the first anchoring element and the second anchoring element comprises a hole.

10. The medical suture of claim 9, wherein the elongate suture wire extends through at least a portion of the hole.

11. A suture deployment device, comprising:
a housing;
a substantially hollow elongate needle comprising a slot extending along a length thereof, wherein the substantially hollow elongate needle is coupled at a proximal end to the housing, and wherein a distal end of the substantially hollow elongate needle comprises a sharpened tip for piercing tissue;
a suture storage cartridge coupled to the housing and adapted to load a suture anchoring element into the proximal end of the substantially hollow elongate needle;
a push rod adapted to retractably extend through the substantially hollow elongate needle; and
a deployment mechanism for retractably extending the push rod though the substantially hollow elongate needle, wherein the push rod is adapted to eject a loaded suture from a distal end of the substantially hollow elongate needle when extended.

12. The suture deployment device of claim 11, wherein the substantially hollow elongate needle comprises a reinforced proximal portion.

13. The suture deployment device of claim 11, wherein the substantially hollow elongate needle comprises a curvature along a longitudinal extent thereof.

14. The suture deployment device of claim 11, wherein the slot is adapted to provide a channel for passing at least a portion of an elongate suture wire during ejection of the suture.

15. The suture deployment device of claim 11, wherein the suture storage cartridge is releasably coupled to the housing.

16. The suture deployment device of claim 11, wherein the substantially hollow elongate needle and the suture storage cartridge are coupled together, and are jointly releas ably coupled to the housing.

17. The suture deployment device of claim 11, wherein suture storage cartridge comprises a spring mechanism.

18. The suture deployment device of claim 11, wherein the suture storage cartridge is adapted to load a suture anchoring element into the proximal end of the substantially hollow elongate needle when the push rod is retracted into the housing.

19. The suture deployment device of claim 11, wherein the suture storage cartridge is adapted to hold a plurality of suture anchoring elements.

20. The suture deployment device of claim 11, wherein the suture storage cartridge is loaded through the substantially hollow elongate needle.

21. The suture deployment device of claim 11, wherein the suture storage cartridge comprises a preloaded, disposable cartridge.

22. The suture deployment device of claim 11, wherein the deployment mechanism comprises a triggering mechanism.

23. The suture deployment device of claim 22, wherein the triggering mechanism comprises at least one of a spring mechanism, a gear mechanism, a lever mechanism, a magnetic mechanism, a hydraulic mechanism, a pneumatic mechanism, and an electrical mechanism.

24. The suture deployment device of claim 23, wherein the electrical mechanism comprises a motor.

25. The suture deployment device of claim 22, wherein the triggering mechanism is manually actuated.

26. A method of deploying a suture, the method comprising the steps of:
providing a suture comprising a first anchoring element and a second anchoring element, with an elongate suture wire extending therebetween;
providing a suture deployment device comprising a substantially hollow elongate needle;
deploying the first anchoring element into a first location of a treatment site of a patient through the substantially hollow elongate needle;
deploying the second anchoring element into a second location of the treatment site through the substantially hollow elongate needle;
cutting the elongate suture wire extending between the first anchoring element and a second anchoring element to create two elongate suture wire portions; and
tying the two elongate suture wire portions together to anchor the first anchoring element and a second anchoring element.

27. The method of claim 26, further comprising loading the suture into the suture deployment device prior to deployment.

28. The method of claim 26, wherein the suture deployment device comprises a triggering mechanism.

29. The method of claim 28, wherein the step of deploying the first anchoring element comprises the steps of:
inserting a distal end of the substantially hollow elongate needle into the treatment site at a first location; and
actuating the triggering mechanism to eject the first anchoring element from the distal end of the substantially hollow elongate needle.

30. The method of claim 29, wherein the step of deploying the second anchoring element comprises the steps of:
removing the distal end of the substantially hollow elongate needle from the treatment site;
inserting the distal end of the substantially hollow elongate needle into the treatment site at a second location; and
actuating the triggering mechanism to eject the second anchoring element from the distal end of the substantially hollow elongate needle.

31. The method of claim 26, wherein the treatment site comprises a hernia or a region of pelvic prolapse.

32. The method of claim 26, further comprising the step of placing a support element within the treatment site.

33. The method of claim 31, wherein the support element comprises a patch for a ventral hernia procedure.

34. The method of claim 32, wherein at least one of the first anchoring element and second anchoring element is deployed along a circumference of the support element.
